(19) United States
(12) Patent Application Publication
(10) Pub. No.: US 2007/0026030 A1
(43) Pub. Date: Feb. 1, 2007

(54) Method of preparing rheological materials for bone and cartilage repair

(75) Inventors: Ken Gill, San Francisco, CA (US); Ken Trauner, San Francisco, CA (US); Francois Genin, Berkeley, CA (US); Ping Luo, Berkeley, CA (US)

Correspondence Address: Berkeley Advanced Biomaterials, Inc., Suite 101, 901 Grayson Street, Berkeley, CA, 94710, US

(73) Assignee: Berkeley Advanced Biomaterials, Inc.

(21) Appl. No.: 11/189,711

(22) Filed: Jul. 27, 2005

(51) Int. Cl. A61F 2/28; A61K 35/32; A61K 33/06

(52) U.S. Cl. 424/423; 623/16.11; 424/682; 424/426; 424/549

(57) ABSTRACT

Methods of mixing delivering biocompatible cement, paste, putty, or gel for bone and cartilage repair are described in this invention. Powder-like solid materials are loaded into a first syringe. Liquids are loaded into one or multiple syringes. The liquids are injected into the first syringe containing the solid materials. To force the liquids through the solid, prevent bubble formation and provide intimate intermixing, the liquids are injected in the very proximity of the plunger end of the syringe containing the solid materials. The first syringe is preferably held vertical with the tip facing up so as to avoid bubble formation that in turn could cause back-pressure build-up and plug the first syringe during injection. The described methods of mixing the liquids with the solids allows to form a rheological paste, cement, putty, or gel in the first syringe. As injection into the human or animal body proceeds, the paste then flows without complications often caused by entrapped bubbles or improper/heterogeneous mixing. The preparation and injection processes can be conducted at temperatures that do not damage live tissue or denature proteins. The paste, cement, putty, or gel can be injected into bone through the cannula by hand or with a pressurizing system. The method reduces the amount of time needed to prepare the paste and load it into the syringe and provides a device that is easily prepared for injection.

### FIELD OF INVENTION

This invention relates to a method of delivering bone cement, bone putty, bone paste or gel for surgical fixation of prostheses in fractured, diseased and osteoporotic bones as well as cartilage; filling of bone voids or cavities; or for the treatment of bone disease, bone tumors or bone cancer. Solid and liquid materials are combined by injecting the liquid into the base of the solid material. The liquid and solids are mixed as the plunger on the syringe is compressed, driving the liquid through the solid material. The paste, cement, putty or gel is then injected into the body prior to hardening. The method is applicable to the rapid production of a viscous paste for delivering materials including metallic, oxide, inorganic, ceramic, organic, polymeric compounds or mixtures of such compounds. The method is useful for shortening the preparation time and facilitating the preparation of the injectable paste in the surgical suite or emergency room. The method is also applicable to the fixation of prostheses to bone for reconstructive surgical procedures in areas such as the hip, knee, shoulder for conditions such as osteoarthritis, rheumatoid arthritis, traumatic arthritis, avascular necrosis, sickle cell anemia, metabolic conditions, fractures of the femoral neck, non-union of fractures of neck and femur, revision of failed arthroplasty procedures. The method is also applicable for the augmentation of fracture fixation in which there is bone loss, comminution or poor bone quality. The method is applicable to the treatment of vertebral conditions with kyphoplasty or vertebroplasty procedures and is also applicable to the treatment of bone tumors, cysts and malignancies. The injection method described in this invention allows pressurization of the liquid through the powder in the syringe starting at the most distal base of the powder adjacent to the plunger. The method thereby reduces the formation of bubbles in the syringe, increases the mixing capacity, and mitigates the potential for plugging the syringe because of bubble-induced back-pressure.

### BACKGROUND OF INVENTION

A number of methods to deliver cement, paste, putty, or gel materials to treat bone ailments have been developed in the past. The numerous techniques developed have reflected the mixing requirements of each material and the methods for use in the operating room. Materials have included methyl methacrylate, calcium sulfates, calcium phosphates, collagen, hyaluronic acid, etc.

The most commonly used bone cement in orthopedic surgery is polymethyl methacrylate (PMMA). To prepare this material, the liquid monomer must be mixed with methyl methacrylate-styrene copolymer to give it the desired cement characteristics. The cement is either prepared externally by mixing the two components in a cup or by pushing the components loaded in a dual-barrel syringe through a cylinder with a helicoidal mixer. After the material is fully mixed, the liquid material is placed either directly into a syringe or caulking gun or as a putty placed manually into the desired defect. Numerous additional PMMA mixing methods exist for mixing the powder material with the liquid solvent prior to injecting into the body. Designed to reduce bubble formation in the final product, these techniques include vacuum mixers, centrifuges, etc, combined with use of caulking gun type apparatus for injection. A typical bone cement injection device has a pistol-shaped body, which supports a cartridge containing bone cement. Patent U.S. Pat. No. 4,994,065 describes an apparatus for dispensing low viscosity semi-fluid material under pressure. The gun includes a cylindrical housing with a plunger and an axially sliding ratchet block concentric with the plunger rod. A pistol grip and lever extend from the housing and the lever engages the ratchet block to advance the cylinder.

U.S. Pat. No. 5,558,136 describes a surgical cement cartridge with piston for ejecting surgical cement at a surgical site. U.S. Pat. No. 5,797,679 describes an apparatus for mixing a two part cement. The cartridge for injecting cement has in inlet coupled to the outlet of the mixing chamber for receiving mixed cement. The mixing chamber has a rotating blade to mix and eliminate voids and to advance the mixed cement into the cartridge.

For filling of bone voids, a trigger activated cartridge system has also been described. According to U.S. Pat. No. 4,969,888 and No. 5,108,404, a cavity can be first formed by compacting cancellous bone inside the bone, into which the bone cement is injected. A caulking gun apparatus is then used to inject pre-packed tubes of material into the created void. A trigger in the gun actuates a spring-loaded ram, which forces pre-packed volume of bone cement in tubes in a viscous condition through a nozzle and into the interior of a bone targeted for treatment. The tubes for injection are filled with material after the material has been mixed to a consistency that allows its placement into tubes and injection. This patent does not describe a technique for mixing the injectable materials.

In U.S. Pat. No. 6,613,054, a new system and method for delivery viscous material into bones, with rate and volume control, was developed to overcome the drawbacks of conventional delivery systems. A specially designed apparatus is necessary. A subcutaneous path is established for introducing material into bone. The instrument comprises a body having a length and a terminus. The body includes markings located along the length at increments from the terminus. The markings allow the physician to gauge the position of the instrument in the subcutaneous path, as material is being tamped into bone. The markers allow the physician to rapidly locate the terminus and estimate the subcutaneous path depth. The terminus of the instrument is advanced through the cannula to urge material residing in the cannula into bone.

The apparatus also includes a nozzle instrument capable of advancement through the subcutaneous cannula into bone and comprising a proximal fitting to couple the nozzle instrument to the delivery device. The nozzle also includes a nozzle bore, through which the material conveyed by the delivery device enters bone at the delivery pressure. The apparatus further includes a stylet capable of advancement into the nozzle bore through the proximal fitting to close the nozzle bore and, with the nozzle instrument. Together, the nozzle and the stylet form a tamping instrument capable of advancement through the subcutaneous cannula to urge residual material from the subcutaneous cannula. Although this apparatus conveys the material at a low delivery pressure, it still requires up to 360 psi to deliver the material.

Recently, calcium phosphate and calcium sulfate bone graft materials have been adopted for use in filling bone voids, augmenting fracture fixation and augmenting reconstructive procedures. These materials are prepared externally to the body by mixing the calcium based powder with a liquid, usually water. The paste or putty materials are then manually manipulated. They are either placed into a syringe and injected into the defect or directly placed manually into the defect.

The calcium materials behave very differently than the non-biologically active PMMA. Materials containing the calcium sulfate have very short setting times. Unlike PMMA which allows 10-15 minutes of mixing and setting time to combine the materials and to inject, these materials may only afford less than one to two minutes to mix and inject. Currently available kits provide the powder material in a bowl or basin. The liquid is added, mixed then placed by finger or spatula into a syringe. The plunger is then attached to the syringe and advanced to inject the material. This technique has numerous disadvantages. Incomplete mixing in the bowl of powder with liquid leads to different setting times and handling characteristics. Significant material may be lost in transfer from bowl to syringe. Valuable injection time is lost in mixing outside the syringe then transferring to the syringe. Capture of air bubbles in the syringe will affect the flow of material and may lead to premature termination of flow.

Numerous designs of specialized syringes exist for combining two materials. Dual chamber syringes are commonly used to combine two materials. U.S. Pat. No. 4,424,057 describes a wet-dry syringe for combining and mixing a liquid and a solid medicament in the same syringe prior to injection. It describes a first vial containing solid or liquid with a second vial that functions as a piston rod. A second rod seal contains a needle that pierces a seal in the first vial thereby mixing the medicaments prior to injection.

U.S. Pat. No. 6,648,852 describes a dispenser for a tissue sealant in which a dry powder is stored in a container having a septum at one end and open end opposite the septum and a movable plug. The powder is retained at the septum end of the container by the movable plug which is displaced and pushed back as the solvent used for reconstituting the powder is introduced through the septum. The second part of the sealant is contained within a second container also with a movable plug. After reconstitution of the first part, a manifold pierce both septums and allows the contents to be dispersed.

U.S. Pat. No. 5,935,101 describes a two compartment type prefilled syringe having a plug separating components e.g. solvent and dry medicament. The syringe mixes components when the plug is displaced to open the by-pass, which is shaped for gentle blending of components. U.S. Pat. No. 6,645,179 describes an injection syringe for use in preparation of vial-handled injection of unstable chemicals/substances. It comprises a multi-chambered cylindrical ampule, a tripartite case, a needle holder, and a plunger.

Solid and liquid mixing devices exist for preparing an injectable material. The dual chamber packets contain a liquid container and a solid container separated by a partition. U.S. Pat. No. 6,544,213 describes a dual compartment mixing and dispensing device suitable for the containment of two separate components that are mixable, upon breaking of a seal that separates the components to provide and instantly mixed dose of medication.

No techniques currently exist for in situ preparation and mixing of a Theological paste within the syringe in a single nondivided chamber.

This invention describes a delivery method for mixing and injecting a Theological paste into tissue that is convenient, efficient, quick and simple to prepare. The technique greatly facilitates intraoperative preparation, eliminated exposure of the powder to handling, eliminates material waste, shortens preparation time and increases the working time available to the doctor for injection. The technique reduces cost by allowing for the use of conventional syringes, needles or cannulas without the need for specialized injection apparatus. The mixing takes place in situ, in the syringe itself. This shortens the handling time and also decreases the potential for external contamination by bacteria, virus or toxic materials. A trained operator can prepare the rheological paste in seconds.

The method uses a pre-packed syringe filled with the calcium based powder and a syringe containing fluid. The procedure involves inserting the needle of the liquid containing syringe in a retrograde fashion to the base of the powder packed syringe adjacent to the plunger. The liquid is then injected into the base (most proximal aspect) of the powder filled syringe. The syringe is then compressed by pushing down on the plunger. The compression forces the fluid antegrade through the powder. Initially no flow occurs through the powder filled tip of the syringe. The fluid mixes with the powder throughout the syringe with compression of the plunger. When the fluid reaches the tip and mixing occurs at the tip, further compression of the plunger will initiate flow of the material from the syringe. At this point, the material may be injected via varied sized cannulas into the bone defect. The procedure can be repeated if several liquids from different syringes must be loaded so as to provide a gradient of composition or a set of multiple products. No complex apparatus and instrument are needed. The procedure can be performed manually at room temperature or can use pre-heated liquids if necessary. If the material in the syringe containing the solid materials is packed somewhat loosely, no pressurizing system is needed to inject the liquid.

### SUMMARY OF INVENTION

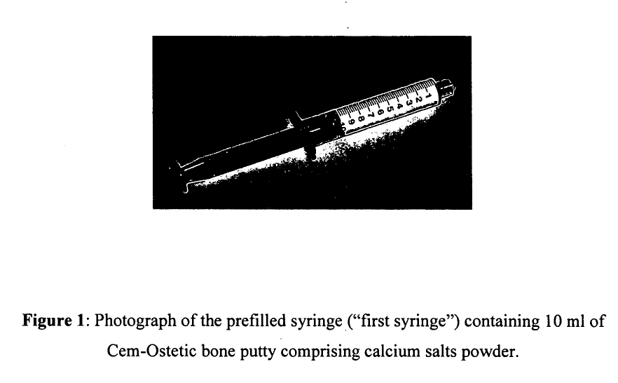

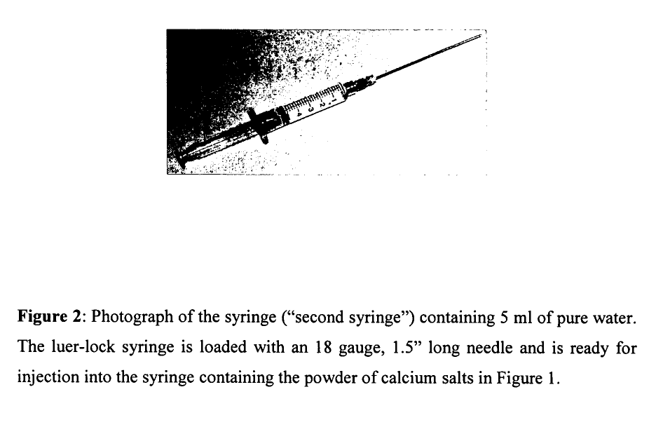

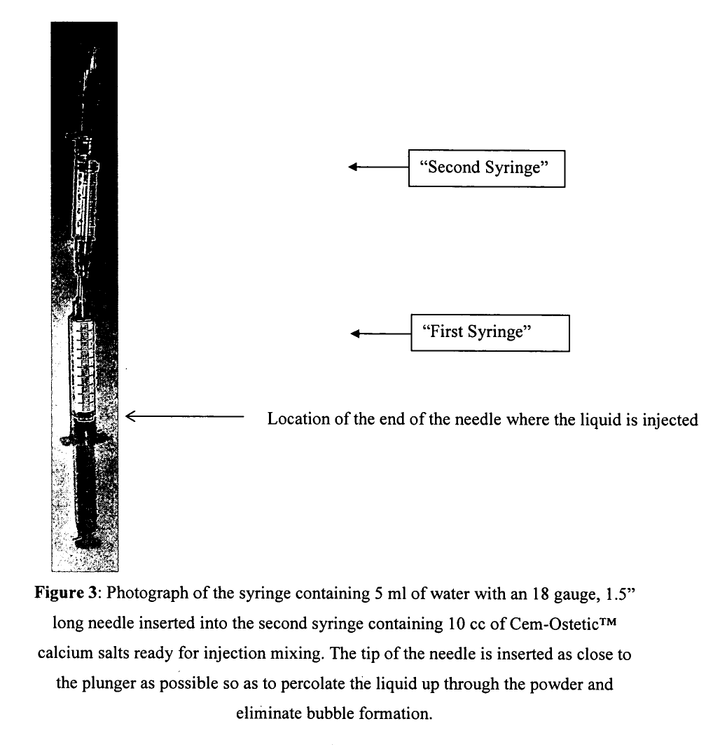

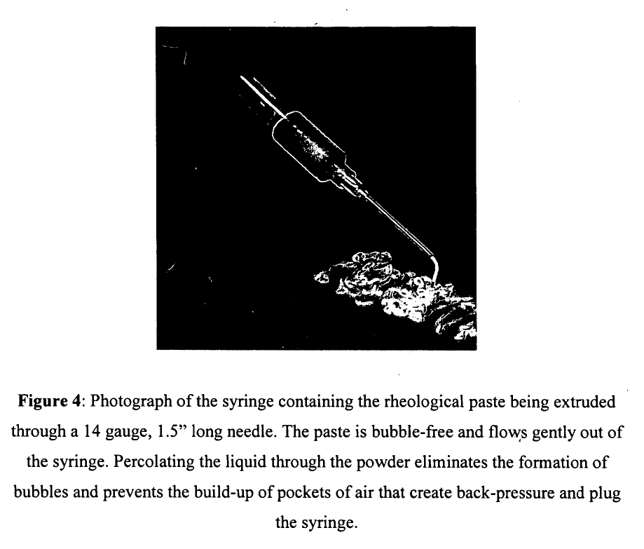

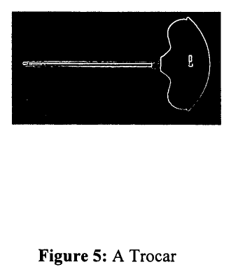

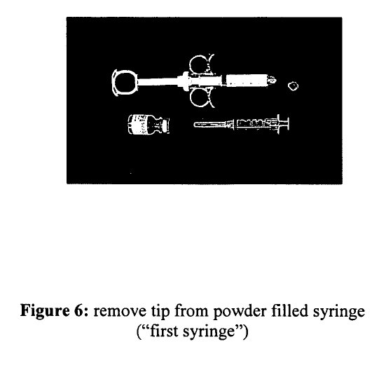

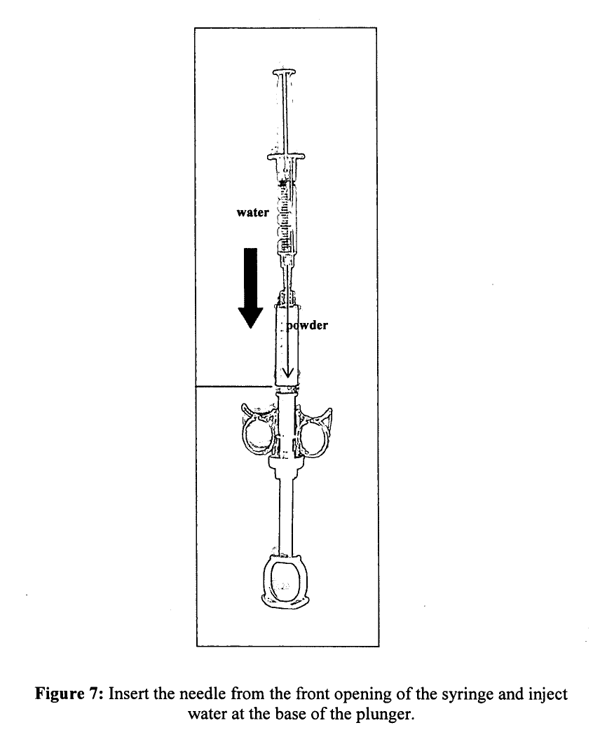

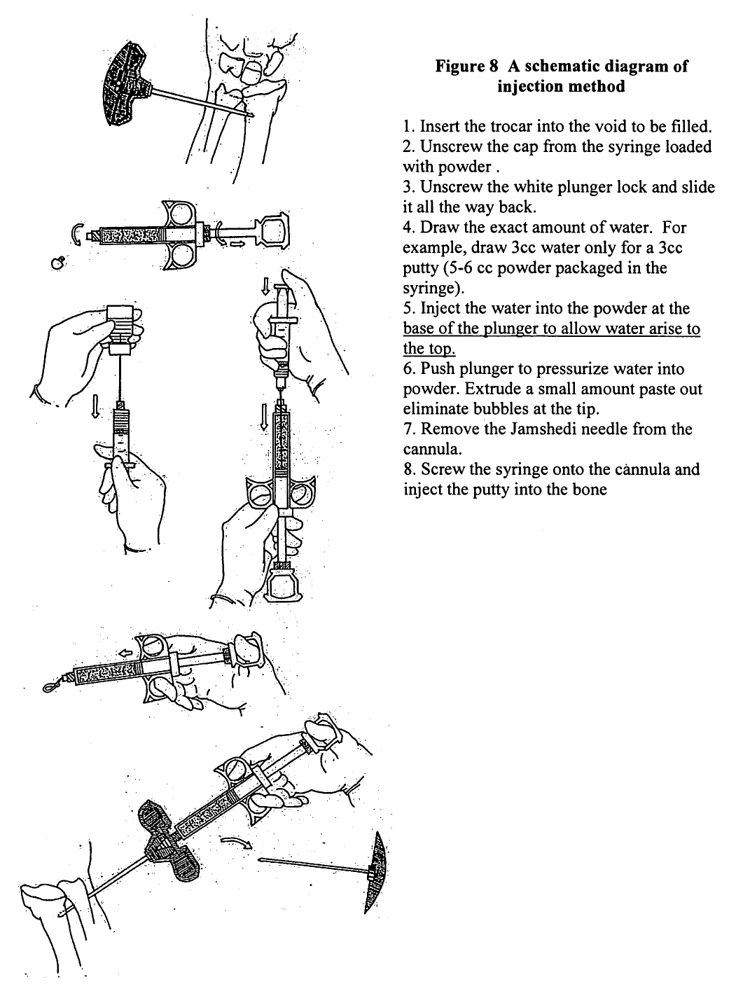

In a specific embodiment of the invention, a biocompatible, resorbable or non-absorbable preformed solid powder, containing calcium salts, bioactive glass, DBM, or protein powder such as bone morphogenetic protein, growth factors, hormones, or polymers, such as PMMA, other conventional homopolymer or copolymer, is pre-packaged in a standard syringe in appropriate capacity ranging from 0.5 ml to 200 ml. All syringes can be disposable if needed. All materials used in the procedure are sterile. Alternatively, the solid powder can be placed in a squeeze bottle or a soft tube of suitable capacity that would serve the role of syringe. A liquid, containing water, aqueous solution (e.g. saline, phosphate buffered solution), bone marrow aspirate, blood, resins, organic hardeners, liquid biomolecules, or drugs is packaged in another syringe in appropriate capacity of ranging from 0.1 ml to 150 ml. The syringe used to measure the liquid volume and inject it is sterile. The liquid can either be pre-packaged in the syringe, pulled from a glass or plastic vial or drawn from the patient prior to the procedure. An example of the syringe kit is shown in FIGS. 1 and 2. The position of the syringe during the mixing procedure is illustrated in FIG. 3. The paste extrusion process and the morphology of the rheological paste flowing through the needle are shown in FIG. 4. During a surgical procedure, the doctor or his/her assistant opens the sterile packages containing the pre-filled syringe of powder, the needle for injecting the bone paste or cement, the syringe of liquid, the needle for liquid injection and the vial of liquid if the liquid is not already pre-filled. The liquid can be drawn into the syringe by aspiration from a vial with a gauge #18 needle if not already pre-filled. The liquid is then injected through the tip of the first needle (as shown in FIG. 3). The tip of the needle must be placed at the base of the syringe close to the surface of the plunger so as to maximize the volume of powder through which the liquid later percolates. Once the liquid is injected, the liquid syringe is removed and discarded. The syringe is then compressed by pushing down on the plunger. The compression forces the fluid antegrade through the powder and auto-mixing of the liquid and powder occurs in situ throughout the syringe. Initially, no flow occurs through the powder filled tip of the syringe. When the fluid reaches the tip and mixing occurs at the tip, further compression of the plunger will initiate flow of the material from the syringe. At this point, the material may be injected into the bone defect. A large bore needle (e.g. #14 gauge spinal needle or cannula) is placed onto the syringe containing the paste. The cannula can establish a subcutaneous path into bone or cartilage. The length of the cannula or bone needle can range from 0.5″ to 12″. The paste is then injected into a bone or cartilage defects or into the cavity of a vertebral body by applying pressure to the plunger of the syringe. It can also be injected into the cavity of the intermedullary canal of the open bone before a prosthetic device is inserted. Whether the surgery involves a hip joint, a tibia, radius or elbow fracture, similar procedures can be conducted. Within a short period of time, the cement, putty or gel solidifies and the operator can close the surgical site.

The method prevents the formation of bubbles or pockets of air in the cartridge of the syringe. The technique greatly facilitates intraoperative preparation, eliminated exposure of the powder to handling, eliminates material waste, shortens preparation time and increases the working time available to the doctor for injection. The technique reduces cost by allowing for the use of conventional syringes, needles or cannulas without the need for specialized injection apparatus.

The preferred injection method of the present invention is directed to use syringes and needles, but it is conceivable that a squeezable bottle, tube, or bag can be filled with solid powder and that a needle can be replace by a flexible tube. As long as the liquid can be injected moves through the solid from the sealed bottom and then gradually percolates to the top, the mixing method allows the proper homogenous mixing of the two ingredients and reduces the potential for plugging the injection tip.

In one embodiment, bone cement, paste, putty, or gel comprising organic or inorganic compounds, can be injected using this method.

In another embodiment, the method delivers material manually without usage of pressurizing instruments.

Features and advantages of the inventions are set forth in the description and figures, as well as in the appended claims.

The term “holding vessel” is a device that can do injection. It can be a syringe. It is also called the “first syringe” in the invention.

The “opening” of a holding vessel can be the tip of a syringe.

A “bone graft material” is a material that can be used to repair bone, to fill a bone void, to repair bone or cartilage defects, to fill an osteoporotic defect, to fill a hole in hard or soft tissue in the body.

The term “cannula” is a tube that can be made by stainless steel or plastics. The “cannula” may or may not contain a sharp end. If it contains a sharp end, it can be also called a trocar or a needle.

The term “a solid component” in the holding vessel is selected from a group of particles, nanoparticles, micronparticles, powder, granules, fragments, or a whole piece of solid that becomes soft when mixed with a liquid component.

### EXAMPLES

### Example A

A self-setting neutral pH bone putty (Cem-Ostetic) comprised of bioresorbable calcium salts powder is loaded into a 10 ml syringe (see FIG. 1). A second syringe is filled with 5 ml water (see FIG. 2).

Step 1: an 18-gauge needle is mounted to the luer-lock tip of a syringe.

Step 2: the syringe is filled with non-pyrogenic USP sterile water.

Step 3: the female cap of a syringe loaded with Cem-Ostetic powder is removed and placed vertical with the tip facing up.

Step 4: the water is injected by inserting the 18-gauge needle through the tip the pre-filled powder syringe and by placing the tip next to the rubber stopper of the plunger.

Step 5: the needle is withdrawn once all the water is transferred into the powder syringe.

Step 6: a 14-gauge bone needle is placed at the end of the syringe containing the Cem-Ostetic paste.

Step 7: the viscous paste is injected to fill up a bone void.

We claim:

1. A method of preparing a viscous bone graft material in an injection device comprising:
a) providing an injection device comprising a holding vessel containing a solid component of a bone graft material;
b) introducing a liquid component of the bone graft material into the holding vessel so as to contact the solid component with the liquid component; and
c) compressing the liquid component and solid component together forming the bone graft material.

2. The method of claim 1 wherein the injection device is a “first syringe”, wherein the “first syringe” further comprises a needle or cannula of gauge 18 or lower for injecting the bone graft material.

3. The method of claim 1 wherein the holding vessel further comprises an opening that permits air to escape, wherein the opening is the tip of the “first syringe”, wherein the holding vessel holds a volume of the bone graft material from claim 1 greater than 0.5 ml.

4. The method of claim 1 wherein the solid component blocks the opening preventing the solid and the liquid component from passing through the opening until after step c in claim 1.

5. The method of claim 1 wherein the bone graft material is selected from the group consisting of one or more Theological fluid, cement, paste, putty, and gel.

6. The method of claim 1 wherein the introducing and the compressing are performed in a single step.

7. The method of claim 1 wherein the bone graft material comprises one or more materials selected from a group consisting of collagen, demineralized bone matrix, hyaluronic acid, polyanhydrides, polyorthoesters, polyglycolic acid, polylactic acid, polylactic acid copolymers, alpha-hydroxycarboxylic acid polyesters, polyglycolide (PGA), poly(L-lactide) (PLLA), ply(D,L-lactide) (PDLLA), poly(lactide-co-glycolide) (PLGA), poly(D,L-lactide-co-trimethylene carbonate), polyhydroxybutyrate (PHB), and poly(anhydride-co-imide).

8. The method of claim 1 wherein the bone graft material is a biocompatible or bioresorbable material.

9. The method of claim 1 wherein the liquid component is introduced into the holding vessel at the site most distal to the opening.

10. The injection device in claim 1 is oriented wherein the opening is positioned higher than the site of the liquid component introduction, wherein the opening is positioned at the highest point of the holding vessel.

11. The injection device in claim 1 is oriented wherein the site of liquid component introduction is positioned at the lowest point of the holding vessel, wherein the liquid component is introduced into the holding vessel with a “second syringe”, wherein the “second syringe” has a needle, wherein the liquid component is introduced by inserting the needle through the wall of the holding vessel in claim 1.

12. The needle of the “second syringe” in claim 11 is inserted through the opening to introduce the liquid component within at least 1 cm of the plunger of the “first syringe”, wherein the “second syringe” has a volume of the liquid component of at least 0.1 ml, wherein the “second syringe” has multiple barrels.

13. The method of claim 1 wherein the solid component is powder-like material, wherein the solid component comprises a material selected from a group consisting of one or more methyl methacrylate, calcium sulfate, calcium phosphates, collagen, fibrin, or hyaluronic acid, demineralized matrix, proteins, or peptides.

14. The method of claim 13 wherein powder-like solid component is a calcium phosphates selected from the group consisting of one or more mono-calcium phosphate, di-calcium phosphate, tri-calcium phosphate, tetra-calcium phosphate, hydroxyapatite, and octa-calcium phosphate.

15. The method of claim 13 wherein the powder-like solid component is a calcium sulfate material selected from the group consisting of one or more alpha-calcium sulfate, beta-calcium sulfate, gamma-calcium sulfate, anhydrous calcium sulfate, hemihydrate calcium sulfate, and dihydrate calcium sulfate.

16. The method of claim 13 wherein the powder-like solid component claim 1 is selected from a group consists of one or more calcium carbonate (calcite or aragonite), calcium citrate, calcium oxide, calcium hydroxide, or sodium chloride.

17. The method of claim 1 wherein the solid component comprises calcium sulfate, hydroxyapatite, and tri-calcium phosphate.

18. The method of claim 1 wherein the solid component comprises synthetic bone morphogenic proteins or natural bone morphogenic proteins, wherein the proteins are selected from the group of one or more BMP-1, -2, -3, -4, -5, -6-, -7, -8, -9, -10.

19. The method of claim 1 wherein the solid component comprises demineralized bone matrix or bone chips.

20. The method of claim 1 wherein the solid component comprises bioactive glass.

21. The method of claim 1 wherein the bone graft material is selected from a group of one or more an amorphous material, a fully crystalline material, a partially crystalline material or a poorly crystalline material.

22. The method of claim 1 wherein the liquid component comprises a liquid selected from a group of one or more water, aqueous solution, bone marrow aspirate, blood, resin, organic hardener, organic monomer and liquid biomolecule.

23. The method of claim 1 wherein the liquid is an aqueous solution.

24. The method of claim 1 wherein the liquid component comprises an organic liquid or inorganic liquid.

25. The method of claim 1 wherein the liquid component is sufficiently fluid or viscous to allow passing through a needle of gauge 22 or smaller and at least 1.25 cm in length.

26. The bone graft material in claim 1 is injected into a patient bone void to promote bone growth.

27. The method in claim 1 consisting of in-situ preparation and mixing of a rheological paste within the syringe in a single non-divided chamber.

28. The liquid component is introduced by a “second syringe” to mix with the solid component by passing through the tip or the opening of the “first syringe”.
